(19) United States
(12) Patent Application Publication
(10) Pub. No.: US 2025/0090570 A1
(43) Pub. Date: Mar. 20, 2025

(54) Method for Prophylaxis and Attenuation of COVID19 and other Inflammatory Microbial Acute Respiratory Disease Syndromes through Modulation of Innate and Adaptive Immunity with Poly ICLC

(71) Applicant: Oncovir, Inc., Stevenson, MD (US)

(72) Inventor: Andres M. Salazar, Winchester, VA (US)

(21) Appl. No.: 18/445,641

(22) PCT Filed: Jun. 18, 2022
(86) PCT No.: PCT/US2022/000012

§ 371 Date: Dec. 1, 2023

Related U.S. Application Data
(60) Provisional application No. 63/259,130, filed on Jun. 21, 2021.

(51) Int. Cl. A61K 31/713; A61K 47/34; A61K 47/38; A61P 31/14

(52) U.S. Cl. CPC A61K 31/713; A61K 47/34; A61K 47/38; A61P 31/14

(57) ABSTRACT

The containment of accidental or intentional epidemic disease outbreaks of pathogens to which our populations have limited or no immunity has thus become one of the principal public health challenges of our time. Methods for clinical administration of pharmaceutical compounds for prevention and attenuation of the inflammatory response to microbial diseases, particularly to the use of double stranded ribonucleic acids (dsRNA). Polyriboinosinic-polyribocytidylic acid stabilized with polylysine and carboxymethylcellulose (Poly-ICLC) converts a virus into the equivalent of an attenuated live-microbe vaccine specific to that microbe, so that Poly-ICLC significantly diminishes infectivity if administered appropriately following infection.

This application is entitled to, and claims the benefit of, priority from U.S. Provisional Application Ser. No. 63/259,130, filed Jun. 21, 2021, which is incorporated herein by reference]

### FIELD AND BACKGROUND OF THE INVENTION

### Field of the Invention

The present invention relates in general to methods for clinical administration of pharmaceutical compounds for prevention and attenuation of the inflammatory response to microbial diseases, particularly to the use of double stranded ribonucleic acids (dsRNA), and more particularly to the use of polyriboinosinic-polyribocytidylic acid stabilized with polylysine and carboxymethylcellulose (Poly-ICLC).

### Background Information

The invention described and claimed herein comprises a novel use of Poly-ICLC to convert a microbial infection into the equivalent of an attenuated live-microbial vaccine specific to that microbe, so that Poly-ICLC significantly diminishes morbidity, mortality, and infectivity if administered appropriately before or following infection.

The real dangers posed by emerging pathogens in an increasingly mobile world have taken on new meaning in the context of bioterrorism. The containment of accidental or intentional epidemic disease outbreaks of pathogens to which our populations have limited or no immunity has thus become one of the principal public health challenges of our time. This challenge becomes even more acute when the populations involved are under chronic stress or illness, as in certain underdeveloped or war-torn regions, or in the case of military personnel involved in protracted military operations in such areas.

It is noted that this problem is not limited to “crisis” infectious diseases. Every year, a new vaccine against influenza must be developed because of mutations from the previous years' strains. Development requires prediction of which strains will be predominant. Because such predictions are imperfect, seasonal flu remains a risk even among those vaccinated, and even so, influenza vaccines remain only partly effective.

A typical approach to reducing these dangers is to develop vaccines against the pathogens. Standard vaccine development, however, requires knowledge of the pathogen and development of a vaccine therefore cannot take place in advance. Moreover, once the pathogen is characterized, the process of vaccine design, production and approval takes time. Thus, the limitations of a biodefense strategy that depends on vaccines alone include lack of immediate protection, limitation to specific pathogens or rapidly mutating variants, and unavailability of vaccine in the case of outbreaks for which the pathogen is unknown or only recently identified.

A glaring example is the recent COVID-19 pandemic. Notwithstanding the enormous “warp speed” effort put into developing a vaccine, it took more than a year before the first vaccine was approved for emergency use. The delay resulted in over five hundred million persons infected worldwide and over 6 million lives lost to date.

In addition, again as illustrated by the Covid-19 experience, virus mutation can make existing vaccines ineffective.

A strategy that augments vaccination (or fills the gap before a vaccine is available) through the—use of antivirals has been advocated, but these also have been only partly effective.

Another approach, not previously reduced to practice in the manner disclosed and claimed herein, would be to harnesses the power of host defenses that have already evolved over billions of years. Double-stranded ribonucleic acids (DsRNAs) are not normally found in mammalian cells, but are components of many viruses or byproducts of viral replication. As a result, they are identified as “foreign” or as pathogen-associated-molecular-patterns (“PAMP”s) by mammalian host defense systems and are potent modulators of the immediate innate immune response as well as of longer-term adaptive immunity, in some ways serving as a bridge between the two systems.

One disadvantage of the use of most PAMPS to activate the immune system is the risk of provoking the severe cytokine storm and lung inflammation induced by some pathogens. This risk has been cited by the U.S. Food and Drug Administration as a reason for refusing or delaying approval of clinical trials of Poly-ICLC for COVID-19. We demonstrate that, in contrast to this common belief, if properly administered as disclosed herein, poly-ICLC preferentially modulates not only antiviral mechanisms but importantly, a relatively unique anti-inflammatory action mediated through the MDA5/interferon (IFN) alpha axis so as to attenuate the counterproductive excess reaction to injury related to cytokines and other tissue factors released not only by viral infection, but by other infectious and non-infectious conditions, while at the same time modulating key elements of adaptive immunity and bridging the gap between early non-specific and longer-term specific immunity.

Poly ICLC is a known compound, described, for example, in U.S. Pat. No. 4,349,538 (Hilton B Levy) and published patent applications U.S. Pat. No. 200610223742 A1 (Andres M. Salazar) and 20040005998 A1 (Andres M. Salazar), all of which are incorporated herein by reference. Importantly, however, the use of Poly-ICLC to produce an anti-inflammatory therapeutic effect on severe acute respiratory distress syndromes has not been previously recognized. Indeed, poly-ICLC has yet to be approved by regulatory agencies such as FDA or EMEA. This is largely because of the generally accepted fear that certain immunomodulators, including poly-IC may aggravate the cytokine storm related to these often fatal infections. A recent salient example of this concern is the initial rejection by the US Food & Drug administration (FDA) of permission to conduct a trial of nasal Poly-ICLC for COVID-19 patients, citing concerns of possible aggravation of the cytokine storm and requiring extensive additional animal safety testing. Consequently, the opportunity to conduct confirmatory clinical trials of protection by nasal Poly-ICLC at the height of the COVID-19 pandemic was lost.

Further, we show here that poly-ICLC can activate the host immune system peri-exposure, without prior knowledge of the specific pathogen. Therefore, poly-ICLC would be effective, even against emerging variants of a pathogen and would be of particular use against COVID-19mutations. Moreover, we show that the required dosages are safe in animals and humans.

### SUMMARY OF THE INVENTION

The foregoing problems are overcome, and other advantages are provided by a process using Poly-ICLC to convert a microbial infection into the equivalent of an attenuated live-virus vaccine specific to that microbe by administering Poly-ICLC to a patient (human or animal) believed to have been exposed to, or at risk of exposure to that microbe for as long as several weeks before that exposure.

The invention disclosed and claimed herein can therefore provide protection against the most serious consequences of a viral infection but, unlike conventional vaccines, can be administered following exposure and can be effective even if the virus has not been identified or characterized, both of which are significant advantages over conventional vaccines.

A further advantage is that POLY-ICLC can be delivered and stored at lower cost and in a greater variety of environments than can conventional vaccines.

These and other objects, features and advantages which will be apparent from the discussion which follows are achieved, in accordance with the invention, by providing a novel process for using Poly-ICLC to convert a virus into the equivalent of an attenuated live-virus vaccine specific to that virus, so as to diminish morbidity, mortality, and infectivity if administered appropriately near the time of infection.

The various features of novelty which characterize the invention are pointed out with particularity in the claims annexed to and forming a part of this disclosure. For a better understanding of the invention, its advantages and objects, reference is made to the accompanying drawings and descriptive matter in which a preferred embodiment of the invention is illustrated.

### BRIEF DESCRIPTION OF THE DRAWINGS

FIG. 1 illustrates the structure of Poly-ICLC (available under tradename “Hiltonol”).

FIG. 2 is a chart reporting the weights of mice exposed to SARS Cov-2, showing the difference between those treated in accordance with the invention and a control group, and reporting survival of mice exposed to SARS Cov-2, showing the difference between those treated in accordance with the invention and a control group.

FIG. 3 is a chart reporting viral loads in the lungs (FIG. 3a) and brains (FIG. 3b) of mice exposed to SARS Cov-2, showing the difference between those treated in accordance with the invention and a control group.

FIGS. 4 and 5 are histological slides illustrating the difference in lung inflammation between control mice treated with saline and mice treated with Poly-ICLC prior to exposure to SARS CoV-2.

FIG. 4 shows several histological slides showing lung inflammation in control mice treated with phosphate buffered saline (PBS) prior to viral challenge with SARS COV2.

FIG. 5 shows several histological slides showing lack of significant inflammation in mice treated with Poly-ICLC prior to viral challenge with SARS COV2.

### DESCRIPTION OF THE PREFERRED EMBODIMENT

Poly-ICLC may be produced and used so as to convert a microbial infection into its own attenuated live-virus vaccine equivalent, thereby significantly diminishing infectivity and serious symptoms if administered appropriately following infection, or prophylactically prior to an anticipated exposure.

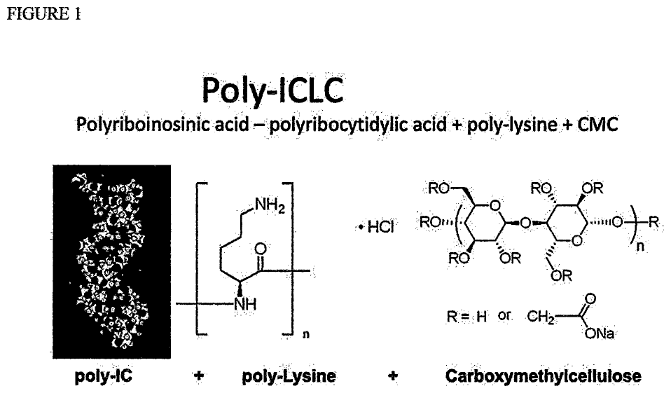

Poly-ICLC is Polyinosinic-Polycytidylic acid stabilized with polylysine and carboxymethylcellulose, a synthetic, stabilized double stranded RNA (dsRNA) therapeutic viral mimic-a pathogen associated molecular pattern (PAMP) that signals through various dsRNA dependent host-defense systems such as the 2′5′OAS, PKR, TLR3, RIG-I and importantly, the cytoplasmic MDA5 helicase, activating multiple elements of innate and adaptive immunity. It is available for experimental clinical use under the tradename Hiltonol® and its structure is illustrated in FIG. 1.

It was initially used as an interferon inducer at very high IV doses in early cancer trials, with transient toxicity and mixed results. Subsequently, lower dose Poly-ICLC was shown to have minimal toxicity with broader immunologic effects, and it has been in extensive preclinical and investigational clinical therapeutic use intramuscularly, intravenously, subcutaneously, and intratumorally, either alone or as a vaccine PAMP-adjuvant in cancer patients.

The mechanisms of action of Poly-ICLC include induction/activation of a ‘natural mix’ of interferons, anti-inflammatory cytokines, chemokines and costimulators, Natural Killer (NK) cells, dendritic cells, CD4 and CD8 T-cells. Most recently it has been shown that many of Poly-ICLCs important actions, including its relative anti-inflammatory and tissue protective effect appears to be mediated largely by preferential activation of the MDA5/IPS1/IFN alpha axis. In contrast to plain Poly-IC which signals primarily through TLR3, Poly-ICLC signals preferentially through the dsRNA-dependent cytoplasmic helicase MDA5, thus activating an alternate IPS1/IFNa pathway that attenuates injury generated by cytokines and other tissue injury factors, DAMPS, TLR-4 ligands, etc. These are typically induced by microbial, ischemic, traumatic and other injury, and are usually the principal source of mortality and morbidity in those conditions. This Poly-ICLC anti-inflammatory effect thus mediates the marked protection seen in ischemic stroke (Gesuete, S N et al. 2016) The differential effect of poly-ICLC versus other PAMPS, including plain Poly-IC, may be partly related to more efficient trafficking of Poly-ICLC through a polylysine-mediated transfection or ‘proton sponge’ effect that bursts the endosome and delivers the dsRNA to the cytosol, where it activates the cytoplasmic dsRNA dependent MDA5 helicase. Poly-IC or Poly-ICLC formulated with lipofectamine, calcium phosphate, nanoparticles or other suitable transfection agent that allows for transport into the cytoplasm and activation of MDA5, RIG-I, OAS, PKR and other cytoplasmic dsRNA dependent systems will accomplish the same end.

The pattern of gene expression or transcriptome induced by Poly-ICLC in humans was shown by Caskey, Lefebvre et al. to closely parallel the pattern induced by an attenuated live virus vaccine against Yellow Fever. Importantly, this pattern of gene activation is markedly different from the pro-inflammatory pattern seen in COVID-19. Based on preclinical studies disclosed below, treatment of COVID-19 patients with poly-ICLC is expected to markedly attenuate the pro-inflammatory COVID pattern, as demonstrated by experiments, the results of which are presented in the drawings.

The present invention shows that the current scientific consensus, including FDA concerns, is mistaken. The invention is based on the recognition that poly-ICLC independently protects against the inflammation and cytokine storm generated by viral as well as certain non-viral microbes such as anthrax infection. It should not only be safe to use in many moderate to advanced infections such as COVID-19, but is expected to be highly beneficial as well. The attenuation of disease by poly-ICLC is thus relatively independent of any direct antiviral effect, as shown by attenuation of infection and protection in multiple animal models in spite of persistent viral titers. The attenuated microbial infection can then allow for development of specific immunity similar to that generated with a live virus vaccine.

In addition, while each strain of virus is relatively unique and it is often impossible to definitively predict the effect of a treatment for one virus or mutated viral strain, many severe infections share inflammation as a common pathogenic mechanism that can be controlled with proper administration of poly-ICLC. While clinical trials are required to fully demonstrate efficacy, Applicant's pre-clinical studies with influenza, coronaviruses, Ebola, anthrax, and other microbes already provide a proof of concept (see below).

### Poly-ICLC Anti-inflammatory Effect

As noted above, there is considerable concern among clinicians and FDA Regulators as to whether POLY-ICLC could exacerbate ongoing virally induced pulmonary inflammation, as is the case with other PAMPS. However, in the case of Poly-ICLC, this is countered by the demonstration of a potent protective therapeutic effect in otherwise rapidly fatal animal models as shown in the drawings, which summarize the results of the following experiment.

In order to test the efficacy of intranasal Poly-ICLC administration for prophylaxis of initial infection the following experiments were conducted. In this study using two animal models, Poly-ICLC was administered intranasally 4 and 3 days before intranasal challenge with SARS-CoV-2. Animal weight and health were monitored daily for 10 days, in addition to viral load and histopathology.

One of the animal models used transgenic mice expressing humanized SARS-COV-2 receptor hACE2 to recapitulate the severe and fatal progression in a subset of patients with COVID-19. Mice challenged nasally with SARS COV-2 (Wuhan) virus develop ultimately fatal upper and lower respiratory tract infection, with extension to brain after day 3. The pathological and immunological manifestations are similar to human COVID-19.

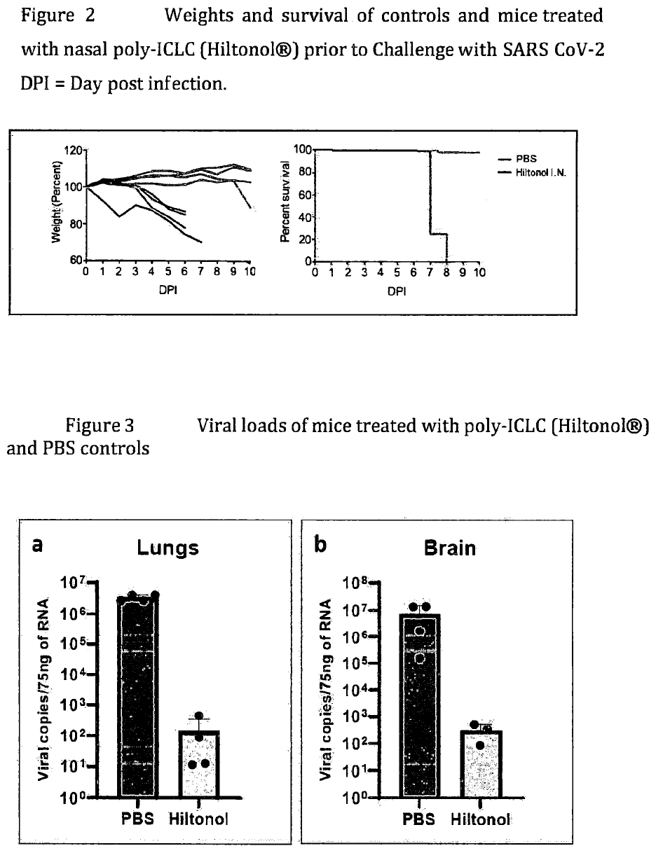

Four mice received two doses of intranasal Poly-ICLC four and three days prior to viral challenge and four mice received intranasal phosphate buffered saline [AM I RIGHT?] (PBS) at the same timepoints. As shown in FIG. 2a, the mice treated with Poly-ICLC maintained or even gained weight, while all control mice lost weight. The control (PBS-treated) group of infected K18-hACE2 mice began to lose weight between 3 and 4 days post-infection (DPI). The mean weight loss in the PBS-treated mice was 20.1%, while the mice treated with Poly-ICLC presented a mean weight gain of 2.5%.

Control mice succumbed to the infection between 7 or 8 DPI while all animals treated with Poly-ICLC survived the challenge as shown in FIG. 2b.

At experiment endpoint (10 DPI) or when PBS-treated animals died (7-8 DPI), lung and brain tissues were collected for viral quantification by qRT-PCR and histopathological analysis. As shown in FIG. 3a & b, PBS-treated mice presented high levels of virus RNA while viral loads in mice treated with Poly-ICLC were several logs lower.

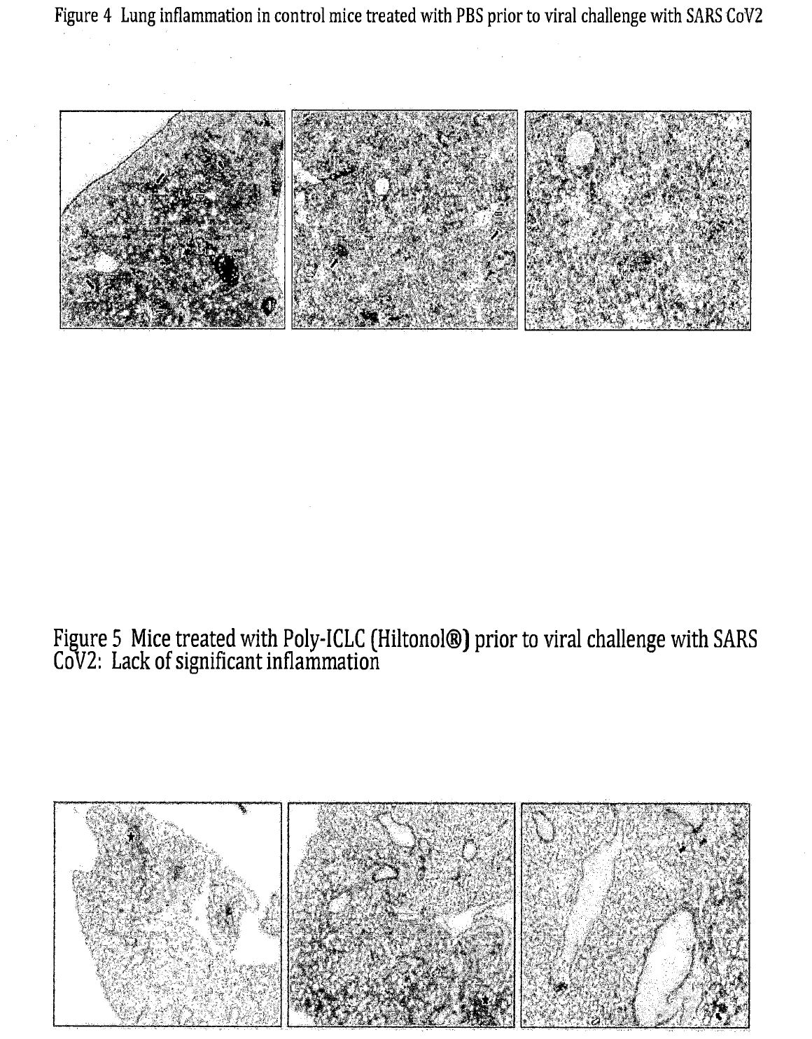

Lung histopathological analysis of the control group revealed an intense inflammatory infiltrate, points of congestion, alveolar collapse, intra-alveolar exudate, and hemorrhagic foci (FIG. 4). In mice treated with Poly-ICLC, the pulmonary architecture was preserved with only mild congestion, focal edema, and a few points of alveolar thickness and collapse (FIG. 5).

These results not only demonstrate the efficacy of the process, but also contradict the common wisdom that Poly-ICLC promotes inflammation.

A second study was conducted using golden Syrian hamsters, a model that mimics milder non-fatal human disease including high viral load and lung inflammation. In this study, all animals survived. However, similar to the above mouse model, a decrease in viral load was seen in the lungs of hamsters treated with Poly-ICLC, in comparison with the control group. At day four post challenge, the lungs from the control group showed a mixed inflammatory infiltrate (mononuclear and polymorphonuclear cells), and an accentuated diffuse congestion with the presence of inflammatory cells in the bronchial space. In hamsters treated with Poly-ICLC only a moderate congestion was observed.

In order to use the invention to treat a human patient, adjustments would need to be made and evaluated by regulatory authorities. The methods for doing so are well known to those of ordinary skill in the art, but would follow the following general pattern.

To establish safety, a phase I safety study would be conducted in healthy volunteers. The following would be one suitable study to evaluate the safety and tolerability of single- and double-dose intranasal Poly-ICLC in healthy adults. The study population could consist of approximately 50 evaluable healthy male and female subjects between 18 and 70 years of age, sequentially randomized into one of 5 cohorts, 3 cohorts receiving one dose of Poly-ICLC or placebo on Day 0, 2 cohorts receiving 2 separate doses of Poly-ICLC or placebo on Day 0 and Day 2, with the dose escalated from.25 mg per nostril to 2 mg/nostril on days 1 and 2. Subjects would be followed for approximately 28 days. Based on ongoing unpublished studies, it is expected that the drug would be well tolerated, with only minor side effects, including transient headaches, nasal irritation, and rhinorrhea and most occurring only once per subject.

To confirm efficacy as a prophylactic, a randomized, placebo controlled double blind study could then be conducted testing Periexposure Prophylaxis of COVID-19 Infection through Host-targeted Activation of Innate and Adaptive Immunity with Nasal Poly-ICLC in Personnel at High Risk for Infection, and comparing nasal Poly-ICLC treatment versus standard prevention for caregivers and other personnel at high-risk for SARS-COV-2 infection. Up to 60 study subjects would be enrolled and randomized at a 2:1 ratio into groups A or B respectively. Enrolled study subjects would receive saline or Poly-ICLC by nasal instillation or nasal spray on days 1, and 2. On days 14 and 15 and again on days 28 and 30, Participants would be followed for a minimum of 45 days if they do not become symptomatically infected, and 60+ days if infected during the study. Comparisons of safety and efficacy will be based on data from concurrently randomized subjects treated at the participating institutions. An independent data and safety monitoring board (DSMB) would actively monitor interim data for safety, efficacy or futility. Nasal instillation of 0.5 ml (0.9 mg) poly-ICLC per nostril (total dose=1.8 mg×2 days=3.6 mg) every two weeks for four cycles would be a suitable dosage, while the control group would receive similar doses of placebo. Patients would be given a set of written instructions to follow, and a diary sheet that they are required to maintain documenting times of treatment and any side effects such as fever or fatigue, rhinitis, nasal congestion, sore throat, or inflammation. The diary will also include entries describing potential coronavirus exposures and dates, and use of masks and hygiene precautions through the course of the treatment and followup. The Primary outcome measure would be the proportion of subjects in each group who develop severe symptomatic COVID-19.

To confirm treatment for infected patients, a suitable protocol would be a phase II trial of attenuation of COVID-19 induced Severe Acute Respiratory syndrome through Poly-ICLC Host-targeted Modulation of Innate and Adaptive Immunity with Nasal Spray Poly-ICLC, a randomized, controlled double blind adaptive phase II pilot study comparing nasal spray Poly-ICLC treatment versus standard care for COVID-19 positive patients with mild to moderate respiratory signs and symptoms. Up to 300 study subjects would be enrolled and randomized at a 2:1 ratio into groups A or B respectively. Enrolled study subjects would receive placebo or Poly-ICLC by nasal instillation on days 1, 2, 8 & 9. Participants would be followed for a minimum of 60 days and comparisons of safety and efficacy based on data from concurrently randomized subjects treated at the participating institutions. An independent data and safety monitoring board will actively monitor interim data for safety, efficacy or futility. The drug would be a colloidal suspension of Poly-ICLC 1.8 mg/ml, administered by nasal instillation of 0.5 ml (0.9 mg) poly-ICLC per nostril (total dose=1.8 mg×2 days Poly-ICLC=Poly-ICLC3.6 mg) via nasal spray. The control group would receive placebo. After the first dose, patients who are not hospitalized can choose to have the study drug instillations administered at the clinic by the clinical staff, or at home as self-administration. The clinical staff will train the study subjects (and person of choice, if applicable) on the proper techniques for the “at home” study drug administration and will observe the first instillation. In addition, patients will be given a set of written instructions to follow, and a diary sheet that they are required to maintain that shows the dates and times when instillations were given, and any side effects such as fever or fatigue, rhinitis, nasal congestion, sore throat, or inflammation. The diary would also include entries describing potential coronavirus exposures and dates, and use of masks and hygiene precautions through the course of the treatment and follow-up. The primary outcome measure would be the proportion of patients who progress to hospitalization for severe Covid-19. A secondary outcome measure would be development and level of antibody and T-cell immunity against SARS COV-2.

It is expected that these studies would confirm the safety of the dosages and effectiveness against severe symptoms. Dosages could be fine-tuned according to protocols known to those of ordinary skill in the art.

Based on the pre-clinical and clinical studies done to date and ongoing, the invention may be used as follows. Note that the steps described below do not depend on advanced knowledge of the pathogen or injury mechanism and therefore can be deployed immediately, without the disadvantage of the delay that prior art vaccines implicate because of their need to be targeted against a specific virus. The experiments described above were conducted using nasal administration. There is, however, no reason why other standard forms of pharmaceuticals could not be used, adapted by techniques known to those of ordinary skill in the art. For example, and not intended to be limiting, Poly-ICLC could be incorporated with an aerosol delivery system, an intravenous delivery system, an intramuscular delivery system or an oral delivery system. Poly-ICLC could also be used as a spray dried powder.

Preferably, nasal spray or aerosolized poly-ICLC is administered in two or more doses spaced 4-72 hours apart, where the dose is in a moderate range (5 to 100 mcg/kg in humans) sufficient to induce measurable but not excessive levels of serum interferon and for unblocking and stimulation of certain interferon and dsRNA inducible enzyme systems, such as the cytosolic dsRNA-dependent MDA5 helicase. As an alternative to nasal administration, the poly-ICLC may be delivered to the lungs via aerosol or may be administered IV IM or orally. Dose cycles may be repeated weekly or twice weekly for up to one year. The preferred dose is from 10 to 50 micrograms per kilogram of body weight.

As its host-targeted anti-inflammatory effects are not dependent on knowing the causative microbe, Poly-ICLC, a safe and effective compound delivered by nasal spray, can be protective against several unknown respiratory microbes or mutants of a known virus including COVID-19, and has significant clinical appeal as an epidemic containment and early treatment tool in both civilian and military applications.

I. For Treatment in Patients who have been recently infected with or without mild to moderate symptomatology, preferably prior to full cytokine storm or acute respiratory distress syndrome or other inflammatory organ failure:

Apply poly-ICLC via nasal instillation or nasal spray, or via aerosol in a single cycle consisting of a dose of 10 to 50 mcg/kg poly-ICLC in single or paired doses spaced 24 to 72 hours apart. Apply poly-ICLC via intramuscular, intravenous, subcutaneous or oral route in a single cycle consisting of a dose of 10 to 50 mcg/kg poly-ICLC in single or paired doses spaced 24 to 72 hours apart.

II. For prevention of infection and/or development of severe symptomatology including cytokine storm or acute respiratory distress syndrome or other inflammatory organ failure in subjects at high risk of exposure but who are not yet infected:

Preferably apply poly-ICLC via nasal instillation or nasal spray, or via aerosol in one or more cycles consisting of a dose of 10 to 50 meg/kg in single or paired doses spaced 24 to 72 hours apart. Cycles may be repeated at 1 to 4 week intervals depending on the incubation period and/or the continued risk of exposure to the offending agent.

As a variation of the treatment methods described above, Poly-ICLC may also be dosed alone as described above, followed by its subsequent dosing in combination with a vaccine if the virus is known and a vaccine is available, one to several times or in combination with a vaccine multiple times in from one to multiple cycles that span a dosing regimen that encompasses at least one month, but could encompass intermittent dosing for up to one year.

Furthermore, we have conducted clinical studies demonstrating that Poly-ICLC alone, administered subcutaneously to normal volunteers will upregulate several hundred genes representing some 10 canonical innate immune pathways and other signaling that play critical roles in the body's natural defenses against a variety of tumors and infections. The pattern of gene activation was very similar to that of a Yellow Fever attenuated live virus vaccine. Poly-ICLC is thus an ‘authentic and reliable’ mimic of certain attenuated viral infections in man.

### Detailed Background of Poly-ICLC, COVID-19, and Other Viral Infections

Paired-Dosing with Poly-ICLC: The exact interplay between dsRNA, IFN and these IFN-inducible systems is not totally elucidated, but the role of dsRNAs such as Poly-ICLC may be bimodal: beginning with induction of IFN related genes and expression of dsRNA dependent systems such as 2′5′OAS, PKR, TLR3, RIG I, MDA-5 and likely others; and followed by their ligation or activation by the dsRNA. Prior studies have suggested that at least two doses of Poly-ICLC administered at about a 24-48 hour interval will markedly increase interferon as well as antiviral action by as much as 100-fold (Marcus and Sekellick 2001) This is also the successful dosing regimen used in glioma clinical trials and in the preclinical influenza and SARS murine studies described below. In this context, the initial dose of Poly-ICLC may be inducing expression of the MDA5 and various other dsRNA-dependent systems described above, while the second dose serves to activate them. One common evasive mechanism used by viruses, including SARS and SARS COV 2, is sequestration or blocking of dsRNA signaling. Exogenous Poly-ICLC may be circumventing this evasion. Additionally, in one sense, the repeated dosing with Poly-ICLC mimics the continual ‘danger signal’ that is typically provided by a replicating virus and that may help the host to differentiate between an incidental exposure to dsRNA and a more genuine threat.

Thus, there has been described a novel process for using Poly-ICLC so as to convert a virus into the equivalent of a live-virus vaccine against that specific virus, thereby significantly diminishing infectivity if administered appropriately following infection or if administered prophylactically to an individual at risk of exposure to the virus.

While a specific embodiment of the invention has been shown and described in detail to illustrate the application of the principles of the invention, it will be understood that the invention may be embodied otherwise without departing from such principles and that various modifications, alternate constructions, and equivalents will occur to those skilled in the art given the benefit of this disclosure. Thus, the invention is not limited to the specific embodiment described herein, but is defined by the appended claims.

### REFERENCES

Caskey, M., F. Lefebvre, . . . , A. Salazar, S. Schlesinger, R. Steinman and R. Sekaly, et al (2011). “Synthetic double stranded RNA reliably induces innate immunity similar to a live viral vaccine in humans” J Exp Med 208.
Christopher M. and J. Wong (2011). “Use of TLR3 receptor agonists against respiratory viral infections.” Anti-inflam & Anti-allergy Agents in Med Chem. 10:327-33
Kato H. Takeuchi O. . . . Akira S, et al, (2006) “Differential roles of MDA5 and RIG-I helicases in the recognition of RNA viruses. “Nature 441 (7089): 101-105.
Gesuete R, Christensen S, Bahjat F, PhD,1 Amy E. B. Packard A. . . . , Salazar AM, Stenzel Poore M. et al. (2016). Cytosolic receptor MDA5 mediates Poly-ICLC preconditioning induced neuroprotection against cerebral ischemic injury. Stroke. 2016 January;47 (1): 262-6. doi: 10.1161/STROKEAHA.115.010329. Epub 2015 Nov. 12.PMID: 265641
Kende, M., J. Paragas and A. M. Salazar (2019). “The efficacy of poly-ICLC against Ebola-Zaire virus (EBOV) infection in mice and cynomolgus monkeys.” Antiviral Res 163:179-184.
Kumaki, Y., A. M. Salazar, M. K. Wandersee and D. L. Barnard (2017). “Prophylactic and therapeutic intranasal administration with an immunomodulator, Hiltonol ((R)) (Poly IC:LC), in a lethal SARS COV-1 infected BALB/c mouse model.” Antiviral Res 139:1-12.
Rockx, B., T. Baas, G. A. Zornetzer, B. Haagmans, T. Sheahan, M. Frieman, M. D. Dyer, T. H. Teal, S.Proll, J. van den Brand, R. Baric and M. G. Katze (2009). “Early upregulation of acute respiratory distress syndrome-associated cytokines promotes lethal disease in an aged-mouse model of severe acute respiratory syndrome coronavirus infection.” J Virol 83 (14): 7062-7074.
Walberg, K., S. Baron, J. Poast, B. Schwartz, L. Izotova, S. Pestka and J. Peterson (2008). “Interferon Protects Mice Against Inhalation Anthrax.” J Interferon & Cytokine Res. 28:597-602
Wong, J., M. Christopher, S. Viswanathan, X. Dai, A. Salazar, L. Sun and M. Wang (2009). “Antiviral Role of Toll-Like Receptor-3 Agonists Against Seasonal and Avian Influenza Viruses.” Current Pharmaceutical Design 15.
Sultan H, Wu J, Kumai T, Salazar AM, Celis E. (2018) Role of MDA5 and interferon-I in dendritic cells for T cell expansion by anti-tumor peptide vaccines in mice. (2017) Cancer Immunol Immunotherapy. https://doi.org/10.1007/s00262-018-2164-6
Sultan H, Salazar AM, & Celis, E (2018) Poly-ICLC, a multifunctional immune modulator for treating cancer. Seminars in Immunology, Review, https://doi.org/10.1016/j.smim.2020.101414

What is claimed is:

1. A method for preexposure prophylaxis and inflammatory symptom attenuation of microbial infection comprising administration of single or paired doses of Poly-ICLC via nasal instillation, spray or aerosol in subjects at risk for infection.

2. A method for inflammatory symptom attenuation of microbial infection comprising administration of single or paired doses of Poly-ICLC via instillation, nasal spray or aerosol in infected patients.

3. The method of claim 1 or claim 2 wherein additional doses of Poly-ICLC in the range of 5 to 100 mcg/kg body weight are administered 4-72 hours apart.

4. The method of claim 1 or claim 2 or claim 3 wherein the doses or the additional doses are in the range of approximately 0.5 to two mg.

5. The method of claims 1 or claim 2 or claim 3 or claim 4 whereby paired doses are administered at one to four week intervals.

6. Use of Poly-ICLC for preparing a medicine for pre-exposure prophylaxis, treatment and/or inflammatory symptom attenuation of microbial infections.

7. Use, according to claim 6 wherein the medicine is administered via instillation, nasal spray or aerosol.

8. Use, according to claim 7, wherein the microbial infection is a SARS-COV-2 infection.

9. A medicine for pre-exposure prophylaxis, treatment and/or inflammatory symptom attenuation of microbial infections, comprising Poly-ICLC.

10. The medicine of claim 9, wherein the Poly-IC is formulated with lipofectamine, calcium phosphate, nanoparticles or other suitable transfection agent that allows for transport into the cytoplasm and activation of MDA5, RIG-I, OAS, PKR and other cytoplasmic dsRNA dependent systems.

11. The method of any of claims 1 through claim 8 wherein the Poly-IC is formulated with lipofectamine, calcium phosphate, nanoparticles or other suitable transfection agent that allows for transport into the cytoplasm and activation of MDA5, RIG-I, OAS, PKR and other cytoplasmic dsRNA dependent systems.

12. Use, according to claims 1-9 in man.

13. Use, according to claims 1-9 in a domestic animal.

14. Use, according to claims 1-9 in wild animals.

15. The method of claim 1 or claim 2 wherein the infection is COVID-19.

16. The method of claim 3 wherein the infection is COVID-19.

17. The method of claim 4 wherein the infection is COVID-19.

18. The method of claim 5 wherein the infection is COVID-19.

19. The method of claim 11 wherein the infection is COVID-19.
